(19) United States
(12) Patent Application Publication
(10) Pub. No.: US 2024/0248016 A1
(43) Pub. Date: Jul. 25, 2024

(54) Hemolysis reagent

(71) Applicant: SHIMADZU CORPORATION, Kyoto-shi (JP)

(72) Inventor: Hiroshi IWATA, Kyoto-shi (JP)

(73) Assignee: SHIMADZU CORPORATION, Kyoto-shi (JP)

(21) Appl. No.: 18/420,433

(22) Filed: Jan. 23, 2024

(30) Foreign Application Priority Data
Jan. 25, 2023 (JP) 2023-9295

(51) Int. Cl. G01N 1/40

(52) U.S. Cl. CPC G01N 1/4044; G01N 2001/4088

(57) ABSTRACT

The present invention mainly aims to provide a reagent that can be used for identification of bacterial species in the same way as known hemolysis reagents (e.g., Lysis buffer), and besides the reagent can also be used for drug susceptibility testing by allowing the hemolysis process without killing the bacteria.

The invention can include, for example, a hemolysis reagent for use in a pretreatment for a clinical microbiological test, comprising a nonionic surfactant, wherein the test comprises a bacterial identification test or a drug susceptibility test.

### TECHNICAL FIELD

The present invention belongs to the field of a clinical microbiology test and compositions therefor. The present invention relates to a hemolysis reagent comprising a surfactant.

### BACKGROUND OF THE INVENTION

Infectious diseases caused by bacteria and other microorganisms continue to be a major threat to humanity. For the treatment of infectious diseases, rapid definitive diagnosis and appropriate drug administration are extremely important. Therefore, it is necessary to accurately and rapidly detect pathogenic microorganisms in specimens from subjects suspected of having infectious diseases and to rapidly determine the drug susceptibility of the causative organisms. In such cases, a clinical microbiology test is indispensable in detecting and understanding these microorganisms. For example, it is known that in patients with sepsis, the survival rate drops to less than 20% in the first 24 hours after onset of the sepsis, and rapid testing of positive blood culture specimens is extremely important for appropriate treatment.

In particular, mass spectrometry (MS) using the matrix-assisted laser desorption/ionization (MALDI) method is becoming an important tool for rapid identification of bacterial species in bacterial testing (MALDI analysis). In the past, direct examination of positive blood culture specimens by MALDI analysis has been difficult due to interference by blood cell components. Various studies have been reported in recent years on methods for direct MALDI analysis of positive blood culture specimens by performing pretreatment such as hemolysis and lysis of bacteria (Non-Patent Document 1 to 4).

### PRIOR ART

### Non-Patent Documents

[Non-Patent Document 1] B. La Scola and D. Raoult, PLOS ONE, vol. 4, no. 11, Article ID e8041, 2009.
[Non-Patent Document 2] W. Moussaoui, B. Jaulhac, A.-M. Hoffmann et al. Clinical Microbiology and Infection, vol. 16, no. 11, pp. 1631.1638, 2010.
[Non-Patent Document 3] O. Liesenfeld, L. Lehman, K.-P. Hunfeld, and G. Kost, European Journal of Microbiology and Immunology, vol. 4, no. 1, pp. 1.25, 2014.
[Non-Patent Document 4] M. Christner, H. Rohde, M. Wolters, I. Sobottka, K. Wegscheider, and M. Aepfelbacher, Journal of Clinical Microbiology, vol. 48, no. 5, pp. 1584. 1591, 2010.

### SUMMARY OF THE INVENTION

### Problem to be Solved by the Invention

The bacterial identification methods described in Non-Patent Document 1 to 4 cause many bacteria in the specimen to die because anionic surfactants are mainly used for pretreatment for hemolysis and lysis of bacteria, and the pH during treatment exceeds 9.0. This pretreatment method is widely used for bacterial identification tests because MALDI analysis is possible as long as the proteins derived from the bacteria in the specimen are present. On the other hand, however, this pretreatment method is difficult to apply to drug susceptibility testing because live bacteria are required for drug susceptibility testing. In other words, although the pretreatment method of this conventional technology can be applied to the bacterial identification test, it is difficult to apply it to the drug susceptibility test, and therefore, the overall speed of the bacterial test has not been sufficiently improved.

The present invention mainly aims to provide a reagent that can be used for identification of bacterial species in the same way as known hemolysis reagents (e.g., Lysis buffer), and besides the reagent can also be used for drug susceptibility testing by allowing the hemolysis process without killing the bacteria.

### Means for Solving the Problem

The present inventor found, as a result of intensive studies, that using a certain nonionic surfactant as a hemolytic agent allows hemolytic treatment without killing the bacteria, thereby solving the above problem of the present invention.

The present invention can include, for example, the following embodiments.
[1] A hemolysis reagent for use in a pretreatment for a clinical microbiological test, comprising a nonionic surfactant, wherein the test comprises a bacterial identification test or a drug susceptibility test.
[2] The hemolysis reagent according to the [1] above, wherein the nonionic surfactant comprises a polyoxyethylene (POE) alkyl ether.
[3] The hemolysis reagent according to the [2] above, wherein the POE alkyl ether comprises a POE (7) alkyl (sec-C₁₁₋₁₅) ether.
[4] The hemolysis reagent according to the [1] above, further comprising a dispersing agent.
[5] The hemolysis reagent according to the [4] above, wherein the dispersing agent comprises a long-chain saturated fatty acid salt or a long-chain unsaturated fatty acid salt.
[6] The hemolysis reagent according to the [5] above, wherein the long-chain unsaturated fatty acid salt comprises an oleate salt.
[7] A pretreatment kit for a clinical microbiology test, comprising the hemolysis reagent according to the [1] above and a dispersing agent to be mixed with the hemolysis reagent.
[8] A pretreatment method for a clinical microbiological test, comprising a step of mixing a culture of a blood sample collected from a subject and the hemolysis reagent according to any one of the [1] to [6] above to obtain a hemolyzed sample.
[9] The pretreatment method according to the [8] above, comprising a step of removing impurities from the mixture of the culture and the hemolysis reagent by filtration or centrifugation.
[10] A clinical microbiology testing method, comprising a step of introducing the hemolyzed sample obtained by the pretreatment method according to the [8] above into a bacterial identification testing system and/or a drug susceptibility testing system.
[11] A pretreatment system for a clinical microbiology test, comprising a means for mixing a culture of a blood sample collected from a subject and the hemolysis reagent according to any one of the [1] to [6] above to obtain a hemolyzed sample.
[12] A clinical microbiology testing system, comprising the pretreatment system according to the [11] above.
[13] The clinical microbiology testing system according to the [12] above, further comprising a bacterial identification testing system or a drug susceptibility testing system.

### Effects of the Invention

The present invention can be used for a bacterial identification test in the same way as known hemolysis reagents (e.g., Lysis buffer), and additionally, it can also be used for a drug susceptibility test and enables rapid diagnosis in the same test because it can perform hemolysis without killing the bacteria in the specimen.

### BRIEF DESCRIPTION OF THE DRAWING

FIG. 1 is a flowchart representing a mode of the clinical microbiology testing method according to an embodiment of the present invention.

FIG. 2 is micrographs showing the results of hemolytic treatment with the hemolysis reagents according to an embodiment of the present invention. The upper figure shows a result of an untreated blood sample, the middle figure shows a result of the hemolytic treatment of Example 1-1, and the lower figure shows a result of the hemolytic treatment of Example 1-2.

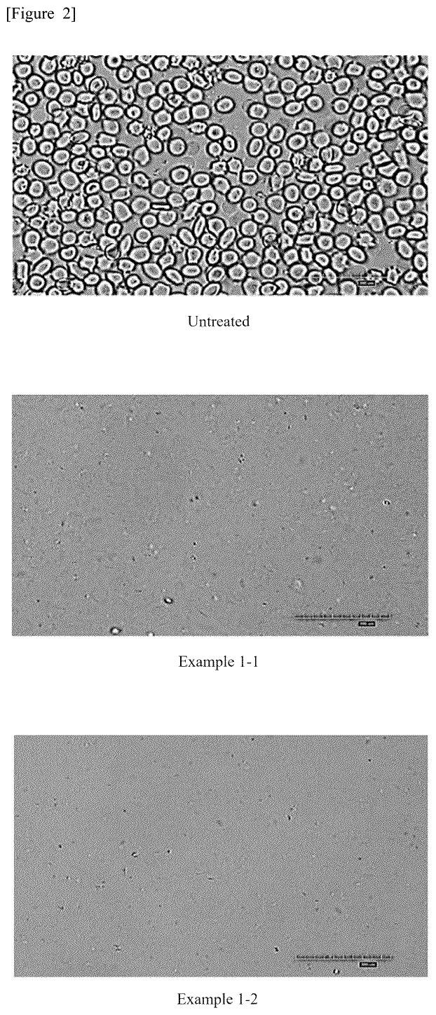

### EMBODIMENT FOR CARRYING OUT THE PRESENT INVENTION

### 1 Hemolysis Reagent for Embodiment of the Present Invention

The hemolysis reagent for the embodiment of the present invention is a hemolysis reagent for use in the pretreatment for a clinical microbiology test, and is characterized in that it comprises a nonionic surfactant and said test comprises a bacterial identification test or a drug susceptibility test. The hemolysis reagent can further comprise, for example, a dispersing agent.

The term “clinical microbiology test” refers to microbiological testing of specimens collected from patients and other subjects, and includes both bacterial identification tests and drug susceptibility tests. It is appropriate for the aforementioned hemolysis reagent to be used for blood specimens.

The term “drug susceptibility test” refers to a test to determine the degree of susceptibility of bacteria, etc. to antimicrobial agents (antibiotics). The aforementioned hemolysis reagent is less damaging to bacteria, etc., and can therefore be suitably used for a pretreatment in the drug susceptibility test.

The term “bacterial identification test” refers to a test to determine the genus and species of bacteria by examining the properties of the test organisms cultured from specimens collected from a subject. The aforementioned hemolysis reagent is preferably used for a pretreatment in the bacterial identification test by mass spectrometry.

Mass spectrometry is not particularly limited, as long as it is an analytical method in which a sample is ionized into gaseous ions using an ion source, and the ionized sample is moved in a vacuum and separated according to its mass-to-charge ratio using electromagnetic force or by time-of-flight difference, and then detected.

Ionization methods include, for example, electron ionization (EI), chemical ionization (CI), field desorption/ionization (FD), fast atom bombardment (FAB), matrix-assisted laser desorption/ionization (MALDI), and electrospray ionization (ESI) methods. The MALDI method is preferred among these methods.

Methods for separating ionized blood samples according to their mass-to-charge ratio include, for example, magnetic field polarization-, quadrupole-, ion trap-, time-of-flight (TOF)-, and Fourier transform ion cyclotron resonance-type separation methods. Among these, TOF-type separation methods are preferred.

Additionally, in the embodiment of the present invention, tandem mass spectrometry (MS/MS), which is a combination of two or more mass spectrometry methods, may be utilized. The mass spectrometry may also be carried out after a sample is separated and purified by gas chromatography (GC) or liquid chromatography (LC) or high-performance liquid chromatography (HPLC).

A mass spectrometer using, for example, a matrix-assisted laser desorption/ionization time-of-flight mass spectrometer (MALDI-TOF MS) is preferred.

### 1.1 Nonionic Surfactants

The aforementioned hemolysis reagent comprises a nonionic surfactant. Such a nonionic surfactant is not particularly limited as long as they can perform hemolysis without killing bacteria in a blood sample. For example, polyoxyethylene (POE) alkyl ethers are suitable. Among these, POE (7) alkyl (sec-C₁₁₋₁₅) ethers are preferred.

Although the concentration of the nonionic surfactant is not particularly limited as long as it is within the range in which blood specimens can be hemolyzed, in the aforementioned hemolysis reagent, for example, a concentration in the range of 0.01% to 20% by weight is appropriate, a concentration in the range of 0.1% to 10% by weight is preferred, and a concentration in the range of 0.2% to 8% by weight is more preferred.

### 1.2 Dispersing Agent

The aforementioned hemolysis reagent can further comprise a dispersing agent. Such a dispersing agent is not particularly limited as long as it has a dispersing effect to prevent aggregation of blood cell components, and it includes, for example, long-chain saturated fatty acid salts and long-chain unsaturated fatty acid salts. The long-chain saturated fatty acid salts can include, for example, laurates, myristates, palmitates, stearates, arachidates, behenates, and lignoserates. The long-chain unsaturated fatty acid salts can include, for example, palmitoleates, oleates, linoleates, linolenates (such as α-linolenates and γ-linolenates), arachidonates, EPA salts, and nerbonates. Among these, oleates are preferred.

Salts of the above (fatty acids) include salts with inorganic bases and salts with organic bases. The salts with inorganic bases can include, for example, sodium salt, potassium salt, magnesium salt, and calcium salt. The salts with organic bases can include, for example, amino acid salts such as arginine salt, ornithine salt, and lysine salt; fumarate salt, acetate salt, and maleate salt. Among these, potassium salt is preferred.

Although the concentration of the dispersing agent is not particularly limited as long as it is within the range that can reduce aggregation of blood cell components, in the aforementioned hemolysis reagent, for example, a concentration in the range of 0.1% to 30% by weight is appropriate, a concentration in the range of 1% to 10% by weight is preferred, and a concentration in the range of 2% to 5% by weight is more preferred.

1.3 Method of Preparing Hemolysis Reagents in Accordance with the Embodiment of the Present Invention

The aforementioned hemolysis reagent can be prepared by dissolving the above components in a solvent (preferably a biocompatible aqueous solution) using a conventional method. Such a biocompatible aqueous solution is usually an aqueous solution in which the salt and/or sugar concentrations are adjusted by sodium and potassium, etc. It can include, for example, saline, phosphate buffered saline (PBS), Tris-buffered saline (TBS), HEPES-buffered saline.

When the aforementioned hemolysis reagent is in the form of an aqueous solution, it is appropriate for the pH to be in the range of 4.0 to 9.0, for example. In particular, it is preferable that the pH be in the range of pH 4.5 to 8.5, and it is more preferable that the pH be in the range of pH 5.0 to 8.0.

### 2 Pretreatment Kit for the Embodiment of the Present Invention

The aforementioned pretreatment kit is a pretreatment kit for clinical microbiology test and is characterized by being equipped with the aforementioned hemolysis reagent. The aforementioned pretreatment kit is preferably equipped with a dispersing agent to be mixed with the aforementioned hemolysis reagent. When equipped with a dispersing agent, the aforementioned pretreatment kit may be of an all-in-one type, in which the dispersing agent is comprised in the aforementioned hemolysis reagent, or it may be separately equipped with a reagent comprising the dispersing agent, instead of the dispersing agent being directly comprised in the hemolysis reagent.

The aforementioned pretreatment kit may further be equipped with, for example, a blood cell removal filter or a bacteria collection filter.

The pore size of the above blood cell removal filter is usually in the range of 1.0 μm to 10 μm, as long as the pore size allows bacteria to pass through and does not allow blood cells or aggregated blood cell components to pass through. Among these, the pore size in the range of 1.5 μm to 7.0 μm is preferred, and the pore size in the range of 2.0 μm to 5.0 μm is more preferred.

The pore size of the above bacteria collection filter should be a pore size that does not allow bacteria to pass through, and is usually 0.5 μm or less, preferably in the range of 0.2 μm to 0.45 μm, and more preferably in the range of 0.22 μm to 0.4 μm.

The materials of each of the above filters can include, for example, cellulose mixed ester (MCE), polyvinylidene fluoride (PVDF), polytetrafluoroethylene (PTFE, including hydrophilic PTFE), hydrophilic PTFE, polyethersulfone (PES, including hydrophilic PES), hydrophilic polyethersulfone, hydrophilic polypropylene (GHP), nylon (NYL), cellulose acetate (CA), polysulfone (PSF), acrylic copolymers, polyamide, polyester, polycarbonate, and nitrocellulose.

The aforementioned pretreatment kit may be equipped with other devices and instruments that can be used for pretreatment of clinical microbiology tests. Such devices or instruments can include, for example, syringes, vacuum pumps, aspirators, compressors, stirring and shaking equipment, centrifuges, centrifuge tubes, test tubes, and blood culture bottles.

### 3 Pretreatment Method for the Embodiment of the Present Invention

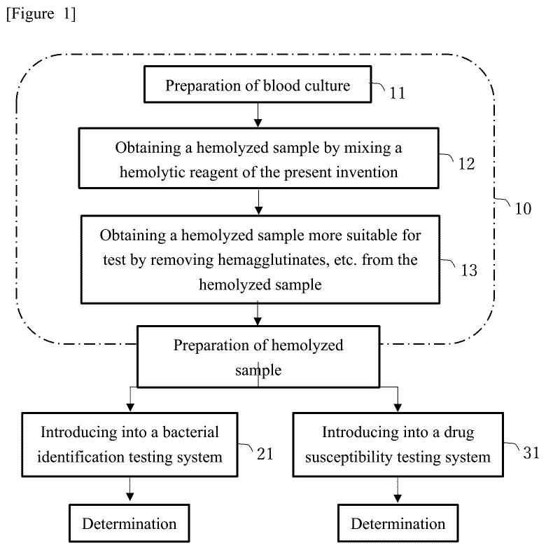

The aforementioned pretreatment method is a pretreatment method for clinical microbiology tests, and is characterized by comprising a step of mixing a culture of a blood sample collected from a subject with a hemolysis reagent to obtain a hemolyzed sample (hemolysis step). One embodiment of the aforementioned pretreatment methods is exemplified in FIG. 1.

The aforementioned subject is not limited. Not only a subject infected with specific bacteria and a healthy subject, but also a subject in which the presence or absence of bacterial infection is unknown, and a subject who may or may not be infected with specific bacteria are widely included.

Conditions, such as temperature and time, for mixing the subject-derived blood culture and the hemolysis reagent to obtain a hemolyzed sample in the hemolysis step 12 is not particularly limited, as long as they are sufficient to destroy red blood cells and release hemoglobin into the blood culture. The temperature can include, for example, the range of 4° C. to 35° C. The treatment time can include, for example, the range of 5 seconds to 10 minutes. The method of mixing the subject-derived blood culture and the hemolysis reagent is not particularly limited, as long as the aforementioned hemolysis reagent is mixed with the entire subject-derived blood culture. The method can include, for example, a method of stirring a container containing a subject-derived blood culture and the aforementioned hemolysis reagent by bringing the container into contact with a stirring and shaking device, a method of stirring by moving the container up and down, a method of stirring with a stirring rod, and a combined method thereof.

The concentration of nonionic surfactant in the subject-derived blood culture mixed with the aforementioned hemolysis reagent is not particularly limited, as long as it is sufficient to destroy red blood cells.

It is appropriate for the subject-derived blood culture mixed with the aforementioned hemolysis reagent to be within the range of pH 4.0 to pH 9.0. In particular, it is preferable to keep the pH within the range of 4.5 to 8.5, and it is more preferable to keep the pH within the range of 5.0 to 8.0. The pH of the blood culture exceeding 9.0 may cause bacteria and other microorganisms to be killed, and the pH lower than 4.0 may cause insufficient hemolysis.

The pretreatment method 10 can include the blood culture step 11 before the hemolysis step. In the step 11, a subject-derived blood culture can be obtained. The subject-derived blood culture can be obtained, for example, by culturing a blood sample collected from a subject for 12 to 48 hours at an incubation temperature of 35° ° C. The blood culture obtained by the blood culture step 11 should preferably be a positive specimen with respect to the growth of microorganisms (bacteria, etc.). More accurate clinical microbiology test results can be obtained by processing the blood culture positive specimen in the pretreatment method 10 and using the processed specimen as a hemolyzed sample. In case that the blood culture is positive, the production of CO₂, H₂, N₂, etc. and consumption of O₂ occur, and then the positivity can be sensed by changes in the concentration and pressure of these gases in the culture vessel. In case that the blood culture is not found to be positive under the aforementioned culture conditions, the culture may be continued.

The pretreatment method 10 can include an impurity removal step 13 after the hemolysis step. The impurity removal step 13 is a process for removing, from the hemolyzed sample obtained through the hemolysis step, those components (e.g., aggregates of blood cell components, etc.) that may be impurities in the clinical microbiology test. The step may be, for example, a process to remove impurities by filtration (filtration process) or a process to remove impurities by centrifugation (centrifugation process). The step can also be a combination of both of these processes (filtration and centrifugation process).

When filtration treatment is employed in the impurity removal step 13, the impurity can be removed more reliably. In the filtration treatment step, for example, blood cell components can be removed from the hemolyzed sample and bacteria in the residue can be recovered by using a filtration filter such as a filter for removing blood cells and a filter for recovering bacteria. The filter for removing blood cells and the filter for recovering bacteria may be used in combination. In the combination use, for example, the filter for removing blood cells can have a pore size that allows bacteria and the like to pass through but does not allow blood cells or aggregated blood cell components to pass through, while the filter for recovering bacteria can have a pore size that does not allow bacteria or the like to pass through. In this case, by passing the hemolyzed sample through the filter for removing blood cells and the filter for recovering bacteria in this order, both removal of blood cell components and collection of bacteria can be performed efficiently.

The filtration treatment method can include, for example, a filtration treatment method in which the hemolyzed sample prepared in the hemolysis step is transferred into a container (e.g., plastic-made container, metal-made container, glass-made container, etc.) equipped with a filtration filter and aspirated or pressurized using a syringe, vacuum pump, aspirator, compressor, or other devices; and a filtration treatment method in which the hemolyzed sample is transferred into a centrifuge tube or test tube equipped with a filtration filter and then filtered using centrifugal force of a centrifuge or the like. From the viewpoint of preventing bacterial contamination, it is preferable to perform the filtration treatment in a closed system. A disposable filter, for example, can be used for the filtration treatment.

The method for recovering microorganism samples such as bacteria in the filtration step may be any method that allows the residue to be detached and recovered from the filtration filter (filter for recovering bacteria) after the filtration step, and can include, for example, a method in which the filter for recovering bacteria is inverted after the filtration step and the recovery liquid or air is injected from the inverted surface (back side of the residue-attached surface). Alternatively, it may be a method of scraping off the microorganism sample trapped on the above filtration filter using a medicine spoon or the like. From the viewpoint of recovery efficiency of bacteria, a method may be used in which a part or all of the above filtration filter is soaked in a solvent and stirred, and then the residue is recovered from the solvent containing the residue by centrifugation or other means.

When centrifugal separation treatment is employed in the impurity removal step 13, impurities can be removed more easily. The centrifugal separation step can be performed using a known centrifuge and centrifugal tubes or the like.

The recovery of the microorganism sample in the centrifugation separation step may be done, for example, by extracting the microorganisms to the supernatant side of the sample during the centrifugation separation treatment, or by sedimentation of the microorganisms. The centrifugation separation step may be performed multiple times in the step, in which case both recovery methods may be used together. For example, after centrifugation treatment using the washing solution (sediment collection), a second centrifugation treatment using the collection solution (supernatant collection) may be performed, or both the first and second and subsequent centrifugations may be performed using the washing solution (sediment collection). From the viewpoint of obtaining a hemolyzed sample with fewer impurities, it is preferable to perform the centrifugation separation step multiple times.

### 4 Clinical Microbiology Testing Method for the of the Present Invention

The aforementioned clinical microbiology testing method is characterized by comprising a step of introducing the hemolyzed sample obtained by the aforementioned pretreatment method into a bacterial identification testing system and/or a step of introducing the same into a drug susceptibility testing system. One embodiment of the aforementioned clinical microbiology testing methods is exemplified in FIG. 1. In the aforementioned clinical microbiology testing method, the hemolyzed sample obtained by the pretreatment method 10 can be used not only for the bacterial identification test but also for the drug susceptibility test, and rapid determination results can be obtained in both tests.

The bacterial identification testing system into which the hemolyzed sample is introduced in step 21 is not particularly limited, as long as the system is capable of identifying bacterial species from the hemolyzed sample. For example, the system can be equipped with a mass spectrometer, and preferably be equipped with a matrix-assisted laser desorption/ionization time-of-flight mass spectrometer (MALDI-TOF MS).

The drug susceptibility testing system into which the hemolyzed sample is introduced in step 31 is not particularly limited, as long as the system is capable of performing drug susceptibility test. Specifically, the drug susceptibility testing system described below can be exemplified.

### 5 Clinical Microbiology Testing System for the Embodiment of the Present Invention

The clinical microbiology testing system is characterized in that it is equipped with a pretreatment system for the embodiment of the present invention described below. The testing system of the present invention may also be equipped with other systems, for example, a bacterial identification testing system and a drug susceptibility testing system.

### 5.1 Pretreatment System for the Embodiment of the Present Invention

The aforementioned pretreatment system is characterized by comprising a means of adding and mixing the aforementioned hemolysis reagent to a culture of blood sample collected from a subject to obtain a hemolyzed sample. The system is not particularly limited, as long as it is capable of realizing the aforementioned pretreatment method, and any known device can be used. It can include, for example, the pretreatment devices described in JP 2022-165506A or JP 2021-65225A.

### 5.2 Bacteria Identification Testing System

The aforementioned testing system can also be equipped with a bacterial identification testing system. The bacterial identification testing system is preferably equipped with a mass spectrometer.

The mass spectrometer is not particularly limited, as long as it is a mass spectrometer that can ionize a residue into gaseous ions using an ion source in the ionization section, and then separate and detect the ionized residue sample according to its mass-to-charge ratio in the mass separation section by moving the sample in a vacuum and using electromagnetic forces or by time-of-flight differences.

The ionization method using an ion source can include, for example, electron ionization (EI), chemical ionization (CI), field desorption/ionization (FD), fast atom bombardment (FAB), matrix-assisted laser desorption/ionization (MALDI), and electrospray ionization (ESI) methods. Any of these methods can be selected as appropriate.

A method of separating ionized blood samples in the mass separation section can include, for example, magnetic field polarization-, quadrupole-, ion trap-, time-of-flight (TOF)-, and Fourier transform ion cyclotron resonance-type separation methods. Any of these methods can be selected as appropriate. Tandem mass spectrometry (MS/MS), which combines two or more mass spectrometry methods, can also be used. Mass spectrometry may also be performed after separation and purification through gas chromatography (GC), liquid chromatography (LC), or high-performance liquid chromatography (HPLC). From the viewpoint of analytical speed and measurable mass range, matrix-assisted laser desorption/ionization time-of-flight mass spectrometry (MALDI-TOF MS) is preferred.

In the above means, if bacteria are detected in the residue sample, data for diagnosing that the subject is highly likely to be infected with bacteria can be obtained. On the other hand, if no bacteria are detected in the residue sample, data for diagnosing that the subject is unlikely to be infected with bacteria can be obtained.

### 5.3 Drug Susceptibility Testing Systems

The aforementioned testing system can also be equipped with a drug susceptibility testing system. The system is not particularly limited, as long as it is capable of performing drug susceptibility testing, and any known system or device can be used. It can include, for example, the testing systems or devices described in JP 2021-083408A or JP 2021-090359A.

### EXAMPLES

The following examples are provided to further illustrate the embodiments of the present invention, but the present invention is not limited in any way to the following examples.

The following raw materials and blood cultures were used.
Nonionic surfactant: POE(7) alkyl (sec-C₁₁₋₁₅) ether (Emulgen 707, manufactured by Kao Co.)
Dispersant: Potassium oleate (19% potassium oleate solution, manufactured by Wako Chemical Co.)
Saline: Japanese Pharmacopoeia Isotonic Sodium Chloride Solution (OTSUKA NORMAL SALINE, manufactured by Otsuka Pharmaceutical Factory, Inc.)
Blood culture: Whole blood of human origin with Staphylococcus aureus (SA) added and incubated in a blood culture bottle (BD Bactec™ 23F Aerobic Resin Bottle P, manufactured by BD) at 35° ° C. for 17 hours.

### The First Example

A hemolysis reagent was prepared by mixing the ingredients listed in Table 1 according to the following procedures (1) through (3) (Examples 1-1 and 1-2).
(1) Two mL of blood culture was placed in a centrifuge tube (13 mL TPP round bottom centrifuge tube) containing 2 mL of the aforementioned hemolysis reagent, shaken and stirred for at least 15 seconds, and centrifuged (1500 g×5 minutes).
(2) The supernatant was removed and 4 mL of resuspension solution was added, shaken and stirred, and then subjected to suction filtration. During the filtration, the specimen was passed through a blood cell removal filter and a bacteria collection filter in this order.
(3) The blood cell removal filter was removed and the bacteria collection filter was inverted, and the bacteria adhering to the filter were collected by injecting the collection solution (4 mL) and air from the inverted side of the bacteria collection filter (back side of the residue adherent surface) to obtain a hemolysed sample.

Each hemolyzed sample obtained was observed under a microscope (RH-2000, manufactured by Hirox Co., Ltd.). The results are shown in FIG. 1. In addition, each hemolyzed sample was placed in Mueller-Hinton medium at room temperature for 24 hours to evaluate the stability of Staphylococcus aureus (SA). The results are shown in Table 1.

TABLE 1

| | | Example 1-1 | Example 1-2 |
|---|---|---|---|
| Ingredients | Nonionic surfactant | 0.5 | 5.0 |
| (wt %) | Dispersant | 3.0 | 3.0 |
| | Saline | 96.5 | 92.0 |

| | | |
|---|---|---|
| SA stability | Survival | Survival |

### The Second Example

A hemolyzed sample was prepared according to the following procedures (1) through (3) (Examples 2-1 and 2-2).
(1) Two mL of blood culture was placed in a centrifuge tube (13 mL TPP round bottom centrifuge tube) containing 2 mL of the aforementioned hemolysis reagent, shaken and stirred for at least 15 seconds, and centrifuged (1500 g×5 minutes).
(2) The supernatant was removed by a pipette or decantation, 4 mL of washing solution was added, shaken and stirred for 5-10 seconds, and centrifuged (1500 g×5 minutes).
(3) The supernatant was removed by a pipette or decantation, and the hemolysed sample was obtained in the following two ways.
(3-1) Four mL of washing solution was added, shaken and stirred for 5 to 10 seconds, centrifuged (1500 g×5 minutes), and the supernatant was removed with a pipette. Four mL of the collection solution was added, shaken and stirred for 5 to 10 seconds (Example 2-1).
(3-2) Four mL of the collection solution was added, shaken and stirred for 5-10 seconds, centrifuged (200g×5 minutes), and 3.5 mL of the supernatant was collected (Example 2-2).

As is evident from FIG. 1, the aforementioned hemolysis reagent showed good hemolytic capacity (Example 1). The stability or viability of the bacteria (Staphylococcus aureus) was also not particularly affected, and good results were obtained (Table 1). The same good results were also obtained for the second Example.

### INDUSTRIAL APPLICABILITY

The present invention contributes to the diagnosis and early prevention or treatment of microbial (e.g., pathogenic bacterial) infections in subjects.

### REFERENCE SIGNS LIST

10 Pretreatment method for the present invention
11 Blood culture step
12 Hemolysis step
13 Impurity removal step
21 Step of introducing a hemolyzed sample into a bacterial identification testing system
31 Step of introducing a hemolyzed sample into a drug susceptibility testing system

1. A hemolysis reagent for use in a pretreatment for a clinical microbiological test, comprising a nonionic surfactant, wherein the test comprises a bacterial identification test or a drug susceptibility test.

2. The hemolysis reagent according to claim 1, wherein the nonionic surfactant comprises a polyoxyethylene (POE) alkyl ether.

3. The hemolysis reagent according to claim 2, wherein the POE alkyl ether comprises a POE (7) alkyl (sec-C₁₁₋₁₅) ether.

4. The hemolysis reagent according to claim 1, further comprising a dispersing agent.

5. The hemolysis reagent according to claim 4, wherein the dispersing agent comprises a long-chain saturated fatty acid salt or a long-chain unsaturated fatty acid salt.

6. The hemolysis reagent according to claim 5, wherein the long-chain unsaturated fatty acid salt comprises an oleate salt.

7. A pretreatment kit for a clinical microbiology test, comprising the hemolysis reagent according to claim 1 and a dispersing agent to be mixed with the hemolysis reagent.

8. A pretreatment method for a clinical microbiological test, comprising a step of mixing a culture of a blood sample collected from a subject and the hemolysis reagent according to claim 1 to obtain a hemolyzed sample.

9. The pretreatment method according to claim 8, comprising a step of removing impurities from the mixture of the culture and the hemolysis reagent by filtration or centrifugation.

10. A clinical microbiology testing method, comprising a step of introducing the hemolyzed sample obtained by the pretreatment method according to claim 8 into a bacterial identification testing system and/or a drug susceptibility testing system.

11. A pretreatment system for a clinical microbiology test, comprising a means for mixing a culture of a blood sample collected from a subject and the hemolysis reagent according to claim 1 to obtain a hemolyzed sample.

12. A clinical microbiology testing system, comprising the pretreatment system according to claim 11.

13. The clinical microbiology testing system according to claim 12, further comprising a bacterial identification testing system or a drug susceptibility testing system.
